SEQUENCE LISTING (4 sequences)

SEQ ID NO: 1 - DNA, 2886 bp, Homo sapiens
  CDS (1)..(2886)
atgttgctgctgctgctactggcgccactcttcctccgccccccgggcgcgggcggggcgcagacccccaacgccacctcagaaggttgccagatcatacacccgccctgggaagggggcatcaggtaccggggcctgactcgggaccaggtgaaggctatcaacttcctgccagtggactatgagattgagtatgtgtgccggggggagcgcgaggtggtggggcccaaggtccgcaagtgcctggccaacggctcctggacagatatggacacacccagccgctgtgtccgaatctgctccaagtcttatttgaccctggaaaatgggaaggttttcctgacgggtggggacctcccagctctggacggagcccgggtggatttccggtgtgaccccgacttccatctggtgggcagctcccggagcatctgtagtcagggccagtggagcacccccaagccccactgccaggtgaatcgaacgccacactcagaacggcgcgcagtgtacatcggggcactgtttcccatgagcgggggctggccagggggccaggcctgccagcccgcggtggagatggcgctggaggacgtgaatagccgcagggacatcctgccggactatgagctcaagctcatccaccacgacagcaagtgtgatccaggccaagccaccaagtacctatatgagctgctctacaacgaccctatcaagatcatccttatgcctggctgcagctctgtctccacgctggtggctgaggctgctaggatgtggaacctcattgtgctttcctatggatccagctcaccagccctgtcaaaccggcagcgtttccccactttcttccgaacgcacccatcagccacactccacaaccctacccgcgtgaaactctttgaaaagtggggctggaagaagattgctaccatccagcagaccactgaggtcttcacttcgactctggacgacctggaggaacgagtgaaggaggctggaattgagattactttccgccagagtttcttctcagatccagctgtgcccgtcaaaaacctgaagcgccaggatgcccgaatcatcgtgggacttttctatgagactgaagcccggaaagttttttgtgaggtgtacaaggagcgtctctttgggaagaagtacgtctggttcctcattgggtggtatgctgacaattggttcaagatctacgacccttctatcaactgcacagtggatgagatgactgaggcggtggagggccacatcacaactgagattgtcatgctgaatcctgccaatacccgcagcatttccaacatgacatcccaggaatttgtggagaaactaaccaagcgactgaaaagacaccctgaggagacaggaggcttccaggaggcaccgctggcctatgatgccatctgggccttggcactggccctgaacaagacatctggaggaggcggccgttctggtgtgcgcctggaggacttcaactacaacaaccagaccattaccgaccaaatctaccgggcaatgaactcttcgtcctttgagggtgtctctggccatgtggtgtttgatgccagcggctctcggatggcatggacgcttatcgagcagcttcagggtggcagctacaagaagattggctactatgacagcaccaaggatgatctttcctggtccaaaacagataaatggattggagggtcccccccagctgaccagaccctggtcatcaagacattccgcttcctgtcacagaaactctttatctccgtctcagttctctccagcctgggcattgtcctagctgttgtctgtctgtcctttaacatctacaactcacatgtccgttatatccagaactcacagcccaacctgaacaacctgactgctgtgggctgctcactggctttagctgctgtcttccccctggggctcgatggttaccacattgggaggaaccagtttcctttcgtctgccaggcccgcctctggctcctgggcctgggctttagtctgggctacggttccatgttcaccaagatttggtgggtccacacggtcttcacaaagaaggaagaaaagaaggagtggaggaagactctggaaccctggaagctgtatgccacagtgggcctgctggtgggcatggatgtcctcactctcgccatctggcagatcgtggaccctctgcaccggaccattgagacatttgccaaggaggaacctaaggaagatattgacgtctctattctgccccagctggagcattgcagctccaggaagatgaatacatggcttggcattttctatggttacaaggggctgctgctgctgctgggaatcttccttgcttatgagaccaagagtgtgtccactgagaagatcaatgatcaccgggctgtgggcatggctatctacaatgtggcagtcctgtgcctcatcactgctcctgtcaccatgattctgtccagccagcaggatgcagcctttgcctttgcctctcttgccatagttttctcctcctatatcactcttgttgtgctctttgtgcccaagatgcgcaggctgatcacccgaggggaatggcagtcggaggcgcaggacaccatgaagacagggtcatcgaccaacaacaacgaggaggagaagtcccggctgttggagaaggagaaccgtgaactggaaaagatcattgctgagaaagaggagcgtgtctctgaactgcgccatcaactccagtctcggcagcagctccgctcccggcgccacccaccgacacccccagaaccctctgggggcctgcccaggggaccccctgagccccccgaccggcttagctgtgatgggagtcgagtgcatttgctttataagtga

SEQ ID NO: 2 - protein, 961 aa, Homo sapiens
MLLLLLLAPLFLRPPGAGGAQTPNATSEGCQIIHPPWEGGIRYRGLTRDQVKAINFLPVDYEIEYVCRGEREVVGPKVRKCLANGSWTDMDTPSRCVRICSKSYLTLENGKVFLTGGDLPALDGARVDFRCDPDFHLVGSSRSICSQGQWSTPKPHCQVNRTPHSERRAVYIGALFPMSGGWPGGQACQPAVEMALEDVNSRRDILPDYELKLIHHDSKCDPGQATKYLYELLYNDPIKIILMPGCSSVSTLVAEAARMWNLIVLSYGSSSPALSNRQRFPTFFRTHPSATLHNPTRVKLFEKWGWKKIATIQQTTEVFTSTLDDLEERVKEAGIEITFRQSFFSDPAVPVKNLKRQDARIIVGLFYETEARKVFCEVYKERLFGKKYVWFLIGWYADNWFKIYDPSINCTVDEMTEAVEGHITTEIVMLNPANTRSISNMTSQEFVEKLTKRLKRHPEETGGFQEAPLAYDAIWALALALNKTSGGGGRSGVRLEDFNYNNQTITDQIYRAMNSSSFEGVSGHVVFDASGSRMAWTLIEQLQGGSYKKIGYYDSTKDDLSWSKTDKWIGGSPPADQTLVIKTFRFLSQKLFISVSVLSSLGIVLAVVCLSFNIYNSHVRYIQNSQPNLNNLTAVGCSLALAAVFPLGLDGYHIGRNQFPFVCQARLWLLGLGFSLGYGSMFTKIWWVHTVFTKKEEKKEWRKTLEPWKLYATVGLLVGMDVLTLAIWQIVDPLHRTIETFAKEEPKEDIDVSILPQLEHCSSRKMNTWLGIFYGYKGLLLLLGIFLAYETKSVSTEKINDHRAVGMAIYNVAVLCLITAPVTMILSSQQDAAFAFASLAIVFSSYITLVVLFVPKMRRLITRGEWQSEAQDTMKTGSSTNNNEEEKSRLLEKENRELEKIIAEKEERVSELRHQLQSRQQLRSRRHPPTPPEPSGGLPRGPPEPPDRLSCDGSRVHLLYK

SEQ ID NO: 3 - DNA, 2823 bp, Homo sapiens
  CDS (1)..(2823)
atggcttccccgcggagctccgggcagcccgggccgccgccgccgccgccaccgccgcccgcgcgcctgctactgctactgctgctgccgctgctgctgcctctggcgcccggggcctggggctgggcgcggggcgccccccggccgccgcccagcagcccgccgctctccatcatgggcctcatgccgctcaccaaggaggtggccaagggcagcatcgggcgcggtgtgctccccgccgtggaactggccatcgagcagatccgcaacgagtcactcctgcgcccctacttcctcgacctgcggctctatgacacggagtgcgacaacgcaaaagggttgaaagccttctacgatgcaataaaatacgggccgaaccacttgatggtgtttggaggcgtctgtccatccgtcacatccatcattgcagagtccctccaaggctggaatctggtgcagctttcttttgctgcaaccacgcctgttctagccgataagaaaaaatacccttatttctttcggaccgtcccatcagacaatgcggtgaatccagccattctgaagttgctcaagcactaccagtggaagcgcgtgggcacgctgacgcaagacgttcagaggttctctgaggtgcggaatgacctgactggagttctgtatggcgaggacattgagatttcagacaccgagagcttctccaacgatccctgtaccagtgtcaaaaagctgaaggggaatgatgtgcggatcatccttggccagtttgaccagaatatggcagcaaaagtgttctgttgtgcatacgaggagaacatgtatggtagtaaatatcagtggatcattccgggctggtacgagccttcttggtgggagcaggtgcacacggaagccaactcatcccgctgcctccggaagaatctgcttgctgccatggagggctacattggcgtggatttcgagcccctgagctccaagcagatcaagaccatctcaggaaagactccacagcagtatgagagagagtacaacaacaagcggtcaggcgtggggcccagcaagttccacgggtacgcctacgatggcatctgggtcatcgccaagacactgcagagggccatggagacactgcatgccagcagccggcaccagcggatccaggacttcaactacacggaccacacgctgggcaggatcatcctcaatgccatgaacgagaccaacttcttcggggtcacgggtcaagttgtattccggaatggggagagaatggggaccattaaatttactcaatttcaagacagcagggaggtgaaggtgggagagtacaacgctgtggccgacacactggagatcatcaatgacaccatcaggttccaaggatccgaaccaccaaaagacaagaccatcatcctggagcagctgcggaagatctccctacctctctacagcatcctctctgccctcaccatcctcgggatgatcatggccagtgcttttctcttcttcaacatcaagaaccggaatcagaagctcataaagatgtcgagtccatacatgaacaaccttatcatccttggagggatgctctcctatgcttccatatttctctttggccttgatggatcctttgtctctgaaaagacctttgaaacactttgcaccgtcaggacctggattctcaccgtgggctacacgaccgcttttggggccatgtttgcaaagacctggagagtccacgccatcttcaaaaatgtgaaaatgaagaagaagatcatcaaggaccagaaactgcttgtgatcgtggggggcatgctgctgatcgacctgtgtatcctgatctgctggcaggctgtggaccccctgcgaaggacagtggagaagtacagcatggagccggacccagcaggacgggatatctccatccgccctctcctggagcactgtgagaacacccatatgaccatctggcttggcatcgtctatgcctacaagggacttctcatgttgttcggttgtttcttagcttgggagacccgcaacgtcagcatccccgcactcaacgacagcaagtacatcgggatgagtgtctacaacgtggggatcatgtgcatcatcggggccgctgtctccttcctgacccgggaccagcccaatgtgcagttctgcatcgtggctctggtcatcatcttctgcagcaccatcaccctctgcctggtattcgtgccgaagctcatcaccctgagaacaaacccagatgcagcaacgcagaacaggcgattccagttcactcagaatcagaagaaagaagattctaaaacgtccacctcggtcaccagtgtgaaccaagccagcacatcccgcctggagggcctacagtcagaaaaccatcgcctgcgaatgaagatcacagagctggataaagacttggaagaggtcaccatgcagctgcaggacacaccagaaaagaccacctacattaaacagaaccactaccaagagctcaatgacatcctcaacctgggaaacttcactgagagcacagatggaggaaaggccattttaaaaaatcacctcgatcaaaatccccagctacagtggaacacaacagagccctctcgaacatgcaaagatcctatagaagatataaactctccagaacacatccagcgtcggctgtccctccagctccccatcctccaccacgcctacctcccatccatcggaggcgtggacgccagctgtgtcagcccctgcgtcagccccaccgccagcccccgccacagacatgtgccaccctccttccgagtcatggtctcgggcctg

SEQ ID NO: 4 - protein, 941 aa, Homo sapiens
MASPRSSGQPGPPPPPPPPPARLLLLLLLPLLLPLAPGAWGWARGAPRPPPSSPPLSIMGLMPLTKEVAKGSIGRGVLPAVELAIEQIRNESLLRPYFLDLRLYDTECDNAKGLKAFYDAIKYGPNHLMVFGGVCPSVTSIIAESLQGWNLVQLSFAATTPVLADKKKYPYFFRTVPSDNAVNPAILKLLKHYQWKRVGTLTQDVQRFSEVRNDLTGVLYGEDIEISDTESFSNDPCTSVKKLKGNDVRIILGQFDQNMAAKVFCCAYEENMYGSKYQWIIPGWYEPSWWEQVHTEANSSRCLRKNLLAAMEGYIGVDFEPLSSKQIKTISGKTPQQYEREYNNKRSGVGPSKFHGYAYDGIWVIAKTLQRAMETLHASSRHQRIQDFNYTDHTLGRIILNAMNETNFFGVTGQVVFRNGERMGTIKFTQFQDSREVKVGEYNAVADTLEIINDTIRFQGSEPPKDKTIILEQLRKISLPLYSILSALTILGMIMASAFLFFNIKNRNQKLIKMSSPYMNNLIILGGMLSYASIFLFGLDGSFVSEKTFETLCTVRTWILTVGYTTAFGAMFAKTWRVHAIFKNVKMKKKIIKDQKLLVIVGGMLLIDLCILICWQAVDPLRRTVEKYSMEPDPAGRDISIRPLLEHCENTHMTIWLGIVYAYKGLLMLFGCFLAWETRNVSIPALNDSKYIGMSVYNVGIMCIIGAAVSFLTRDQPNVQFCIVALVIIFCSTITLCLVFVPKLITLRTNPDAATQNRRFQFTQNQKKEDSKTSTSVTSVNQASTSRLEGLQSENHRLRMKITELDKDLEEVTMQLQDTPEKTTYIKQNHYQELNDILNLGNFTESTDGGKAILKNHLDQNPQLQWNTTEPSRTCKDPIEDINSPEHIQRRLSLQLPILHHAYLPSIGGVDASCVSPCVSPTASPRHRHVPPSFRVMVSGL

(19) United States
(12) Patent Application Publication
(10) Pub. No.: US 2006/0216749 A1
(43) Pub. Date: Sep. 28, 2006

(54) Chimeric gaba receptor

(76) Inventors: Karine Alfonsine Astrid Smans, Wilrijk (BE); Henricus Jacobus Maria Gijsen, Breda (NL)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/569,760

(22) PCT Filed: Sep. 3, 2004
(86) PCT No.: PCT/EP04/52029

§ 371 Date: Feb. 24, 2006

(30) Foreign Application Priority Data
Sep. 12, 2003 (WO) PCT/EP03/10263

(51) Int. Cl. G01N 33/53; C12N 5/06; C07K 14/705; C07H 21/04; C12P 21/06; A61K 31/542

(52) U.S. Cl. 435/7.1; 530/350; 435/69.1; 435/320.1; 435/325; 536/23.5; 514/224.5

(57) ABSTRACT

The present invention provides an isolated GABA_{B} receptor protein comprising at least one GABA_{B}R1a subunit and at least one GABA_{B}R2a subunit, characterized in that said GABA_{B} receptor has one high affinity agonist binding site and one low affinity agonist binding site. In particular the isolated recombinant GABA_{B} receptor protein expressed by the hGABA_{B}R1a/GABA_{B}R2 CHO cell line deposited at the Belgian Coordinated Collections of Microorganisms (BCCM) as CHO-K1 h-GABA-b R1a/R2 clone on Aug. 22, 2003 with the accession number LMBP 6046CB. It is thus an object of the present invention to provide the hGABA_{B}R1a/GABA_{B}R2 CHO cell line deposited at the Belgian Coordinated Collections of Microorganisms (BCCM) as CHO-K1 h-GABA-b R1a/R2 clone on Aug. 22, 2003 with the accession number LMBP 6046CB.

The invention also provides the use of the aforementioned cell line in a method to identify GABA_{B} receptor agonists using a functional or a binding assay. In particular in a radioligand-binding assay comprising the use of radiolabeled agonists such as for example ³H-GABA or ³H-baclofen.

In a particular embodiment the present invention provides the use of the aforementioned GABA_{B} receptor in a method to identify a high affinity GABA_{B} receptor agonist using a functional or a binding assay. In particular in a radioligand-binding assay comprising the use of radiolabeled agonists such as for example ³H-GABA or ³H-baclofen. Alternatively, the aforementioned binding assays are performed on cellular extracts, in particular cellular membrane preparations of the aforementioned cells.

The present invention provides a novel method to identify substances that are agonists of GABA_{B} receptors, using a ³H-GABA binding assay in recombinant GABA_{B}R1a/R2 receptor expressing cells.

### BACKGROUND OF THE INVENTION

GABA (γ-amino-butyric acid) is the most widely distributed amino acid inhibitory neurotransmitter in the central nervous system (CNS) activating two distinct families of receptors; the ionotropic GABA_{A} and GABA_{C} receptors for fast synaptic transmissions, and the metabotropic GABA_{B} receptors governing a slower synaptic transmission.

GABA_{B} receptors are members of the superfamily of seven transmembrane G-protein coupled receptors that are coupled to neuronal K⁺ or Ca²⁺ channels. Presynaptic GABA_{B} receptor activation has generally been reported to result in the inhibition of Ca²⁺ conductance, leading to a decrease in the evoked release of neurotransmitters. Post-synaptically the major effect of GABA_{B} receptor activation is to open potassium channels, to generate post-synaptic inhibitory potentials.

The expression of GABA_{B} receptors is widely distributed in the mammalian neuronal axis, with particularly high levels in the molecular layer of the cerebellum, interpeduncular nucleus, frontal cortex, olfactory nuclei, thalamic nuclei, temporal cortex, raphe magnus and spinal cord. GABA_{B} receptors are also present in the peripheral nervous system, both on sensory nerves and on parasympathetic nerves. Their ability to modulate these nerves give them potential as targets in disorders of the lung, GI tract and bladder (Belley et al., 1999, Biorg. Med. Chem. 7:2697-2704).

A large number of pharmacological activities have been attributed to GABA_{B} receptor activation, such as for example, analgesia, hypothermia, catatonia, hypotension, reduction of memory consolidation and retention, and stimulation of insulin, growth hormone and glucagon release (see Bowery, 1989, Trends Pharmacol. Sci. 10:401-407 for a review). It is well accepted that GABA_{B} receptor agonists and antagonists are pharmacologically useful in indications such as stiff man syndrome, gastroesophogeal reflux, neuropathic pain, incontience and treatment of cough and cocaine addiction. For example, the GABA_{B} receptor agonist baclofen has been shown to reduce transient lower esophagal sphincter relaxations (TLESR) and is accordingly useful in the treatment of reflux as most episodes of reflux occur during TLESR. However, the current GABA_{B} receptor agonists, such as baclofen, are relatively non-selective and show a variety of undesirable behavioural actions such as sedation and respiratory depression. It would be desirable to develop more GABA_{B} receptor agonists with an improved selectivety and less of the aforementioned undesirable effects.

Current methods of drug discovery generally involve assessing the biological activity of tens or hundreds of thousands of compounds in order to identify a small number of those compounds having a desired activity against a particular target, i.e. High Throughput Screening (HTS). In a typical HTS related screen format, assays are performed in multi-well microplates, such as 96, 384 or 1536 well plates, putting certain constrains to the setup of the assay to be performed including the availability of the source materials (i.e membrane preparations of cells expressing the recombinant GABA_{B} receptor). HTS related screens are preferably performed at room temperature with a single measurement for each of the compounds tested in the assay, requiring short cycle times, with a reproducible and reliable output.

Present in vitro screens to identify compounds as agonists of the GABA_{B} receptor, either rely on natural, less abundant resources such as binding assays in rat brain membranes or consist of functional screening assays, such as for example Ca²⁺ responses, c-AMP responses and effects on Ca²⁺ and K⁺ channels performed in cells expressing a recombinant GABA_{B} receptor. In some of these functional assays the GABA_{B} receptors may be co-expressed with G-proteins, e.g. Gα16 or Gqi5 or the chimeric G-protein G αq-z5, increasing G-protein coupling (Bräauner-Osborne & Krogsgaard-Larsen, 1999, Br. J. Pharmacol. 128:1370-1374). However, a GABA_{B} agonist binding assay that would further reduce the HTS cycle time and the resources for biochemicals such as recombinant proteins, is currently unavailable.

The present invention describes the development of a Chinese Hamster Ovary (CHO) cell line co-expressing the human GABA_{B} receptor subunits GABA_{B}R1a and GABA_{B}R2, which were surprisingly found to demonstrate agonist binding in radioligand binding experiments. In addition, the present inventors demonstrated that the hGABA_{B}R1a/GABA_{B}R2 CHO cell line has one high affinity and one low affinity agonist binding site in the recombinant expressed GABA_{B} receptor. Hence the hGABA_{B}R1a/GABA_{B}R2 CHO cell line provided by the present invention not only allows compound screening, but also provides a useful tool to characterize the nature of the compound-receptor interaction.

### SUMMARY OF THE INVENTION

The present invention provides an isolated GABA_{B} receptor protein comprising at least one GABA_{B}R1a subunit and at least one GABA_{B}R2 subunit, characterized in that said GABA_{B} receptor has one high affinity agonist binding site and one low affinity agonist binding site. In particular the isolated recombinant GABA_{B} receptor protein expressed by the hGABA_{B}R1a/GABA_{B}R2 CHO cell line deposited at the Belgian Coordinated Collections of Microorganisms (BCCM) as CHO-K1 h-GABA-b R1a/R2 clone on Aug. 22, 2003 with the accession number LMBP 6046CB. It is thus an object of the present invention to provide the hGABA_{B}R1a/GABA_{B}R2 CHO cell line deposited at the Belgian Coordinated Collections of Microorganisms (BCCM) as CHO-K1 h-GABA-b R1a/R2 clone on Aug. 22, 2003 with the accession number LMBP 6046CB.

The invention also provides the use of the aforementioned cell line in a method to identify GABA_{B} receptor agonists using a functional or a binding assay. In particular in a radioligand-binding assay comprising the use of radiolabeled agonists such as for example ³H-GABA or ³H-baclofen.

The invention further provides a method to identify GABA_{B} receptor agonists, comprising contacting the aforementioned cell line with a test compound and measuring the binding of said test compound to the GABA_{B} receptor. In particular the method consists of a radioligand binding assay, comprising exposing the aforementioned cells to a labelled agonist of GABA_{B} in the presence and absence of the test compound and measure the binding of the labelled ligand to the cells according to the invention, where if the amount of binding of the labelled ligand is less in the presence of the test compound, then the compound is a potential agonist of the GABA_{B} receptor.

It is also an object of the present invention to provide a method to identify a high affinity GABA_{B} receptor agonist, said method comprising contacting the aforementioned cells with the radiolabeled agonist selected from the group consisting of GABA, baclofen and 3-aminopropylphosphinic acid (3-APPA a.k.a APMPA), in the presence and absence of the test compound and measure the binding of the labelled ligand to the cells according to the invention, where if the amount of binding of the labelled ligand to the high affinity binding site is less in the presence of the test compound, then the compound is a potential high affinity agonist of the GABA_{B} receptor.

Alternatively, the aforementioned binding assays are performed on cellular extracts, in particular cellular membrane preparations of the hGABA_{B}R1a/GABA_{B}R2 CHO cell line deposited at the Belgian Coordinated Collections of Microorganisms (BCCM) as CHO-K1 h-GABA-b R1a/R2 clone on Aug. 22, 2003 with the accession number LMBP 6046CB.

In another embodiment the present invention provides a method to identify a GABA_{B} receptor agonist, said method comprising contacting the aforementioned cell line with a compound to be tested and determine whether the compound activates a GABA_{B} receptor functional response in said cells. In particular the functional response consists of modulation of the activity of ion channels or of intracellular messengers as explained hereinafter.

This and further aspects of the present invention will be discussed in more detail hereinafter.

### BRIEF DESCRIPTION OF THE DRAWING

FIG. 1 GTPγ35S-binding upon stimulation of membranes by GABA expressed as the percentage of maximal GABA stimulation, in the presence and absence of the positive allosteric modulator CGP7930>

FIG. 2 Displacement of ³H-GABA by agonists (baclofen, GABA & APMPA) and antagonists (SCH50911 & CGP54626)

FIG. 3 Reproducible agonist IC₅₀ values (n=5) independent of membrane preparations.

FIG. 4 Two sided ³H-GABA agonist binding curve in the presence or absence of 10 μM CGP54626 (a) or JNJ 4309747-AAD (b).

### DETAILED DESCRIPTION

For the purposes of describing the present invention: GABA_{B}R1a or h GABA_{B}R1a as used herein refers to the human GABA_{B} receptor subunit known as GABA_{B}R1a in Kaupmann et al, 1998, Proc. Natl. Acad. Sci. USA 95:14991-14996, the amino acid sequence (SEQ ID No.:2) of which can be found at GenBank Accession no. AJ225028, as well as to its mammalian orthologs. GABA_{B}R1a also refers to other GABA_{B} receptor subunits that have minor changes in amino acid sequence from those described hereinbefore, provided those other GABA_{B} receptor subunits have substantially the same biological activity as the subunits described hereinbefore. A GABA_{B}R1a subunit has substantially the same biological activity if it has an amino acid sequence that is at least 80% identical to, preferably at least 95% identical to, more preferably at least 97% identical to, and most preferably at least 99% identical to SEQ ID No.: 2 and has a Kd or EC50 for GABA, GABA_{B} receptor agonists such as for example baclofen and gabapentin or GABA_{B} receptor antagonists such as for example CGP54626A, SCH 50911, saclofen and phaclofen, that is no more than 5-fold greater than the Kd or EC50 of a native GABA_{B} receptor for GABA or the same GABA_{B} receptor agonist or GABA_{B} receptor antagonist.

GABA_{B}R2 as used herein refers to the human GABA_{B} receptor subunit known as GABA_{B}R2 in White et al., 1998, Nature 396:679-682, the amino acid sequence (Seq Id NO.: 4) of which can be found at GenBank accession no. AF058795 as well as to its mammalian orthologs. GABA_{B}R2 also refers to other GABA_{B} receptor subunits that have minor changes in amino acid sequence from those described hereinbefore, provided those other GABA_{B} receptor subunits have substantially the same biological activity as the subunits described hereinbefore. A GABA_{B}R2 subunit has substantially the same biological activity if it has an amino acid sequence that is at least 80% identical to, preferably at least 95% identical to, more preferably at least 97% identical to, and most preferably at least 99% identical to SEQ BD No.: 4 and has in combination with a GABA_{B}R1 subunit a Kd or EC50 for GABA, GABA_{B} receptor agonists such as for example baclofen and gabapentin or GABA_{B} receptor antagonists such as for example CGP54626A, SCH 50911, saclofen and phaclofen, that is no more than 5-fold greater than the Kd or EC50 of a native GABA_{B} receptor for GABA or the same GABA_{B} receptor agonist or GABA_{B} receptor antagonist.

The Kd and EC50 values of the native GABA_{B} receptor is determined using the methods known to a person skilled in the art, in particular using competition binding studies on tissue preparations such as for example described in Cross & Horton, 1987 Eur.J.Pharmacol. 141(1): 159-162. Briefly, crude synaptic membranes are prepared by homogenisation of whole brain, centrifugation (30 000×g, 20 min.) and extensive washing. Total binding is measured by incubation of the membranes with ³H-GABA or ³H-baclofen, while non-specific binding is measured in the presence of 100 μM baclofen. Upon removal of unbound ligand by filtration, filters are counted in a β-counter or a Topcount Harvester (Packhard). For competition experiments the binding occurs in the presence of increasing concentration of unlabeled compound.

It is thus an object of the present invention to provide an isolated GABA_{B} receptor protein formed by at least one GABA_{B}R1a and at least one GABA_{B}R2 subunit further characterized in that said isolated GABA_{B} has both a high and a low affinity agonist binding site. In a further embodiment this isolated GABA_{B} receptor is a functional GABA_{B} receptor expressed by a cell, wherein said cell does not normally express the GABA_{B} receptor. Suitable cells which are commercially available, include but are not limited to L-cells, HEK-293 cells, COS cells, CHO cells, HeLa cells and MRC cells, in particular CHO cells wherein the GABA_{B} receptor protein comprises at least one GABA_{B}R1a subunit encoded by the oligonucleotide sequence consisting of SEQ ID No.1 and at least one GABA_{B}R2 subunit encoded by the oligonucleotide sequence consisting of SEQ ID No.3. In a more particular embodiment the isolated GABA_{B} receptor according to the invention, consists of the receptor protein expressed by the hGABA_{B}R1a/GABA_{B}R2 CHO cell line deposited at the Belgian Coordinated Collections of Microorganisms (BCCM) as CHO-K1 h-GABA-b R1a/R2 clone 20 on Aug. 22, 2003 with accession number LMBP 6046CB.

“Functional GABA_{B} receptor”refers to a GABA_{B} receptor formed by co-expression of GABA_{B}R2 and GABA_{B}R1a in a cell, wherein said cell does not normally express the GABA_{B} receptor, most preferably resulting in a heterodimer of GABA_{B}R2 and GABA_{B}R1a, where the functional GABA_{B} receptor mediates at least one functional response when exposed to the GABA_{B} receptor agonist GABA. Examples of functional responses are: pigment aggregation in Xenopus melanophores, negative modulation of cAMP levels, coupling to inwardly rectifying potassium channels, mediation of late inhibitory postsynaptic potentials in neurons, increases in potassium conductance, decreases in calcium conductance, MAPKinase activation, extracellular pH acidification, and other functional responses typical of G-protein coupled receptors. One skilled in the art would be familiar with a variety of methods of measuring the functional responses of G-protein coupled receptors such as the GABA_{B} receptor (see, e. g., Lerner, 1994, Trends Neurosci.17: 142-146 [changes in pigment distribution in melanophore cells]; Yokomizo et al., 1997, Nature 387: 620-624 [changes in cAMP or calcium concentration; chemotaxis]; Howard et al., 1996, Science 273: 974-977 [changes in membrane currents in Xenopus oocytes]; McKee et al., 1997, Mol. Endocrinol. 11: 415-423 [changes in calcium concentration measured using the aequorin assay]; Offermanns & Simon, 1995, J. Biol. Chem. 270: 15175-15180 [changes in inositol phosphate levels]). Depending upon the cells in which heteromers of GABA_{B}R1a and GABA_{B}R2 are expressed, and thus the G-proteins with which the functional GABA_{B} receptor thus formed is coupled, certain of such methods may be appropriate for measuring the functional responses of such functional GABA_{B} receptors. It is well within the competence of one skilled in the art to select the appropriate method of measuring functional responses for a given experimental system.

The term “compound”, “test compound”, “agent” or “candidate agent” as used herein can be any type of molecule, including for example, a peptide, a polynucleotide, or a small molecule that one whishes to examine for their activity as GABA_{B} receptor agonist, and wherein said agent may provide a therapeutic advantage to the subject receiving it. The candidate agents can be administered to an individual by various routes, including, for example, orally or parenterally, such as intravenously, intramuscularly, subcutaneously, intraorbitally, intracapsularly, intraperitoneally, intrarectally, intracisternally or by passive or facilitated absorption through the skin, using for example a skin patch or transdermal iontophoresis, respectively. Furthermore the compound can be administered by injection, intubation or topically, the latter of which can be passive, for example, by direct application of an ointment, or active, for example, using a nasal spray or inhalant, in which case one component of the composition is an appropriate propellant. The route of administration of the compound will depend, in part, on the chemical structure of the compound. Peptides and polynucleotides, for example, are not particular useful when administered orally because they can be degraded in the digective tract. However, methods for chemically modifying peptides, for example rendering them less susceptible to degradation are well know and include for example, the use of D-amino acids, the use of domains based on peptidomimetics, or the use of a peptoid such as a vinylogous peptoid.

The agent used in the screening method may be used in a pharmaceutically acceptable carrier. See, e.g., Remington's Pharmaceutical Sciences, latest edition, by E.W. Martin Mack Pub. Co., Easton, Pa., which discloses typical carriers and conventional methods of preparing pharmaceutical compositions that may be used in conjunction with the preparation of formulations of the agents and which is incorporated by reference herein.

Cells

As already outlined above, the present invention provides a cell line stably transfected with expression vectors that direct the expression of the GABA_{B} receptor subunits GABA_{B}R1a and GABA_{B}R2 as defined hereinbefore. In particular CHO cells transfected with said expression vectors. Such expression vectors are routinely constructed in the art of molecular biology and may involve the use of plasmid DNA and appropriate initiators, promoters, enhancers and other elements, which may be necessary, and which are positioned in the correct orientation, in order to allow for protein expression. Generally, any system or vector suitable to maintain, propagate or express polynucleotides to produce a polypeptide in a host may be used. The appropriate nucleotide sequence, i.e. the polynucleotide sequences encoding either the human GABA_{B}R1a or GABA_{B}R2 subunit as defined hereinbefore, may be inserted into an expression system by any of a variety of well-known and routine techniques such as for example those set forth in Current Protocols in Molecular Biology, Ausbel et al. eds., John Wiley & Sons, 1997.

In a particular embodiment the CHO cells according to the invention are cotransfected with the commercially available expression vectors pcDNA3.1 comprising the polynucleotide sequences encoding for human GABA_{B}R1a (SEQ ID No.: 1) and human GABA_{B}R2 (SEQ ID No.: 3) respectively. More preferably the present invention provides a hGABA_{B}R1a/GABA_{B}R2 CHO cell line deposited at the Belgian Coordinated Collections of Microorganisms (BCCM) as CHO-K1 h-GABA-b R1a/R2 clone 20 on Aug. 22, 2003 with the accession number LMBP 6046CB. This cell line is characterized in that the functional GABA_{B} receptor in this CHO cell line has both a low and a high affinity binding site for GABA_{B} receptor agonist. Using the cell line according to the invention, will not only allow compound screening, but also provides a useful tool for the characterization of the nature of the compound-receptor interaction, i.e. does it interact with the low or high affinity agonist binding site of the GABA_{B} receptor.

For further details in relation to the preparation of nucleic acid constructs, mutagenesis, sequencing, introduction of DNA into cells and gene expression, and analysis of proteins, see for example, Molecular Cloning: a Laboratory Manual: 2nd edition, Sambrook et al., 1989, Cold Spring Harbor Laboratory Press.

Assays

The present invention also provides an assay for a compound capable of interacting with the functional GABA_{B} receptor of the present invention, which assay comprises: providing the GABA_{B} receptor expressed by the hGABA_{B}R1a/GABA_{B}R2 CHO cell line of the present invention, contacting said receptor with a putative binding compound; and determining whether said compound is able to interact with said receptor.

In one embodiment of the assay, the receptor or subunits of the receptor may be employed in a binding assay. Binding assays may be competitive or non-competitive. Such an assay can accommodate the rapid screening of a large number of compounds to determine which compounds, if any, are capable of binding to the polypeptides.

Within this context, the present invention provides a method to identify whether a test compound binds to an isolated GABA_{B} receptor protein of the present invention, and is thus a potential agonist or antagonist of the GABA_{B} receptor, said method comprising;

a) contacting cells expressing a functional GABA_{B} receptor, wherein such cells do not normally express the GABA_{B} receptor, with the test compound in the presence and absence of a compound known to bind the GABA_{B} receptor, and

b) determine the binding of the test compound to the GABA_{B} receptor using the compound known to bind to the GABA_{B} receptor as a reference.

Binding of the test compound or of the compound known to bind to the GABA_{B} receptor, hereinafter also referred to as reference compound, is assessed using art-known methods for the study of protein-ligand interactions. For example, such binding can be measured by employing a labeled substance or reference compound. The test compound or reference compound can be labeled in any convenient manner known in the art, e.g. radioactively, fluorescently or enzymatically. In a particular embodiment of the aforementioned method, the compound known to bind to the GABA_{B} receptor, a.k.a. the reference compound is detectably labeled, and said label is used to determine the binding of the test compound to the GABA_{B} receptor. Said reference compound being labeled using a radiolabel, a fluorescent label or an enzymatic label, more preferably a radiolabel. In a more particular embodiment, the present invention provides a method to identify whether a test compound binds to an isolated GABA_{B} receptor protein, said method comprising the use of a compound known to bind to the GABA_{B} receptor, wherein said reference compound is selected from the group consisting of ³H-GABA, ³H-baclofen, ³H-3-APPA, ³H-CGP542626 and ³H-SCH50911.

Subsequently, more detailed assays can be carried out with those compounds found to bind, to further determine whether such compounds act as agonists or antagonists of the polypeptides of the invention.

Thus, in a further embodiment the present invention provides a method to identify GABA_{B} receptor agonists said method comprising,
a) exposing cells expressing a functional GABA_{B} receptor, wherein such cells do not normally express the GABA_{B} receptor, to a labeled agonists of GABA_{B} in the presence and absence of the test compound, and
b) determine the binding of the labeled agonist to said cells,
   where if the amount of binding of the labeled agonist is less in the presence of the test compound, then the compound is a potential agonist of the GABA_{B} receptor. As already specified for the general binding assay above, the binding of the GABA_{B} receptor agonists is assessed using art-known methods for the study of protein-ligand interactions. The label is generally selected from a radioactive label, a fluorescent label or an enzymatic label, in particular a radiolabel wherein the agonist is selected from the group consisting of ³H-GABA, ³H-baclofen and ³H-3-APPA.

Similarly, the present invention provides a method to identify GABA_{B} receptor antagonists said method comprising,
a) exposing cells expressing a functional GABA_{B} receptor, wherein said cells do not normally express the GABA_{B} receptor, to a labeled antagonist of GABA_{B} in the presence and absence of the test compound, and
b) determine the binding of the labeled antagonist to said cells,

where if the amount of binding of the labeled antagonist is less in the presence of the test compound, then the compound is a potential antagonist of the GABA_{B} receptor. As already specified for the general binding assay above, the binding of the GABA_{B} receptor antagonists is assessed using art-known methods for the study of protein-ligand interactions. The label is generally selected from a radioactive label, a fluorescent label or an enzymatic label, in particular a radiolabel wherein the antagonist is selected from the group consisting of ³H-CGP542626 and ³H-SCH50911.

In an alternative embodiment of the present invention, the aforementioned binding assays are performed on a cellular composition, i.e a cellular extract, a cell fraction or cell organels comprising a GABA_{B} receptor as defined hereinbefore. More in particular, the aforementioned binding assays are performed on a cellular composition, i.e. a cellular extract, a cell fraction or cell organels comprising a GABA_{B} receptor as defined hereinbefore, wherein said cellular composition, i.e. cellular extract, cell fraction or cell organels, is obtained from cells expressing a functional GABA_{B} receptor, wherein said cells do not normally express the GABA_{B} receptor. More preferably, the cellular composition, i.e. cellular extract, cell fraction or cell organels, is obtained from the hGABA_{B}R1a/GABA_{B}R2 CHO cell line deposited at the Belgian Coordinated Collections of Microorganisms (BCCM) as CHO-K1 h-GABA-b R1a/R2 clone 20 on Aug. 22, 2003 with the accession number LMBP 6046CB.

It is accordingly, an object of the present invention to provide a method for identifying a compound as a GABA_{B} receptor agonist or antagonist, said method comprising;
a) administering the compound to a cellular composition of cells expressing a functional GABA_{B} receptor, wherein said cells do not normally express the GABA_{B} receptor, in the presence of a detectably labeled agonist or antagonist of the GABA_{B} receptor; and
b) determine the binding of the labeled agonist or antagonist to said cellular composition,

where if the amount of binding of the labeled agonist or antagonist is less in the presence of the test compound, then the compound is a potential agonist respectively antagonist of the GABA_{B} receptor.

As already specified for the general binding assay above, the binding of the GABA_{B} receptor agonist or antagonist is assessed using art-known methods for the study of protein-ligand interactions. The label is generally selected from a radioactive label, a fluorescent label or an enzymatic label, in particular a radiolabel wherein the agonist is selected from the group consisting of ³H-GABA, ³H-baclofen and 3H-3-APPA and the antagonist is selected from the group consisting of ³H-CGP542626 and ³H-SCH50911. In a more specific embodiment the aforementioned binding assays are performed on a cellular composition consisting of the membrane fraction of cells according to the invention, in particular on membrane fractions of the hGABA_{B}R1a/GABA_{B}R2 CHO cell line deposited at the Belgian Coordinated Collections of Microorganisms (BCCM) as CHO-K1 h-GABA-b R1a/R2 clone 20 on Aug. 22, 2003 with the accession number LMBP 6046CB, using one or more of the aforementioned radiolabeled agonsist and/or antagonists.

In a further embodiment the present invention provides a functional assay for identifying compounds that modulate the GABA_{B}-recepor activity in the cells according to the invention. Such an assay is conducted using the cells of the present invention, i.e. cotranfected with the human GABA_{B}R1a and human GABA_{B}R2 subunits. The cells are contacted with at least one reference compound wherein the ability of said compound to modulate the GABA_{B}-receptor activity is known. Thereafter, the cells are contacted with a test compound and determined whether said test compound modulates the activity of the GABA_{B} receptor compared to the reference compound. A “reference compound” as used herein refers to a compound that is known to bind and/or to modulate the GABA_{B} receptor activity.

A compound or a signal that “modulates the activity” of a polypeptide of the invention refers to a compound or a signal that alters the activity of the polypeptide so that it behaves differently in the presence of the compound or signal than in the absence of the compound or signal. Compounds affecting modulation include agonists and antagonists. An agonist of the GABA_{B} receptor encompasses a compound such as GABA, baclofen and 3-APPA which activates GABA_{B} receptor function. Alternatively, an antagonist includes a compound that interferes with GABA_{B} receptor function. Typically, the effect of an antagonist is observed as a blocking of agonist-induced receptor activation. Antagonists include competitive as well as non-competitive antagonists. A competitive antagonist (or competitive blocker) interacts with or near the site specific for agonist binding. A noncompetitive antagonist or blocker inactivates the function of the receptor by interacting with a site other than the agonist interaction site.

In one embodiment the present invention provides a method for identifying compounds that have the capability to modulate GABA_{B} receptor activity, said method comprising;

a) contacting cells expressing a functional GABA_{B} receptor, wherein said cells do not normally express a functional GABA_{B} receptor, with at least one reference compound, under conditions permitting the activation of the GABA_{B} receptor;

b) contacting the cells of step a) with a test compound, under conditions permitting the activation of the GABA_{B} receptor, and
c) determine whether said test compound modulates the GABA_{B} receptor activity compared to the reference compound.

Methods to determine the capability of a compound to modulate the GABA_{B} receptor activity are based on the variety of assays available to determine the functional response of G-protein coupled receptors (see above) and in particular on assays to determine the changes in potassium currents, changes in calcium concentration, changes in cAMP and changes in GTPγS binding. Conditions permitting the activation of the GABA_{B} receptor generally known in the art, for example in case of antagonist screening these conditions comprise the presence of a GABA_{B} receptor agonist in the assay system. Typical GABA_{B} receptor agonists used in these activity assays are GABA, baclofen or 3-APPA. More particular in the GTPγS assay as outlined herein below, GABA is used to activate the GABA_{B} receptor in order to assess the capability of a test compound to inactivate the GABA_{B} receptor protein.

In the aforementioned assay an increase of GTPγS binding in the presence of the test compound is an indication that the compound activates the GABA_{B} receptor activity, and accordingly that said test compound is a potential agonist of the GABA_{B} receptor protein. A decrease of GTPγS binding in the presence of the test compound is an indication that the compound inactivates the GABA_{B} receptor protein and accordingly that said test compound is a potential antagonist of the GABA_{B} receptor protein.

Particularly preferred types of assays include binding assays and functional assays which may be performed as follows:

Binding Assays

Over-expression of the GABA_{B} receptor expressed by the hGABA_{B}R1a/GABA_{B}R2 CHO cell line of the present invention may be used to produce membrane preparations bearing said receptor (referred to in this section as GABA_{B} binding receptor for convenience) for ligand binding studies. These membrane preparations can be used in conventional filter-binding assays (eg. Using Brandel filter assay equipment) or in high throughput Scintillation Proximity type binding assays (SPA and Cytostar-T flashplate technology; Amersham Pharmacia Biotech) to detect binding of radio-labelled GABA_{B} ligands (including ³H-GABA, ³H-baclofen, ³H-3-APPA, ³H-CGP542626, ³H-SCH50911) and displacement of such radio-ligands by competitors for the binding site. Radioactivity can be measured with Packard Topcount, or similar instrumentation, capable of making rapid measurements from 96-, 384-, 1536-microtitre well formats. SPA/Cytostar-T technology is particularly amenable to high throughput screening and therefore this technology is suitable to use as a screen for compounds able to displace standard ligands.

Another approach to study binding of ligands to GABA_{B} binding receptor protein in an environment approximating the native situation makes use of a surface plasmon resonance effect exploited by the Biacore instrument (Biacore). GABA_{B} binding receptor in membrane preparations or whole cells could be attached to the biosensor chip of a Biacore and binding of ligands examined in the presence and absence of compounds to identify competitors of the binding site.

Functional Assays

Since GABA_{B} receptors belong to the family G-protein coupled receptors that are coupled to GIRK (inward rectifying potassium channels), potassium ion flux should result on activation of these receptors. This flux of ions may be measured in real time using a variety of techniques to determine the agonistic or antagonistic effects of particular compounds. Therefore, recombinant GABA_{B} binding receptor proteins expressed in the cell lines of the present invention can be characterised using whole cell and single channel electrophysiology to determine the mechanism of action of compounds of interest. Electrophysiological screening, for compounds active at GABA_{B} binding receptor proteins, may be performed using conventional electrophysiological techniques and when they become available, novel high throughput methods currently under development.

Given the presynaptic effect of GABA_{B} receptor activation on Ca²⁺ channels, in an alternative functional screen the modulatory effect of a compound is assessed through the changes in intracellular calcium. Calcium fluxes are measurable using several ion-sensitive fluorescent dyes, including fluo-3, fluo4, fluo-5N, fura red and other similar probes from suppliers including Molecular Probes. The inhibition of calcium influx as a result of GABA_{B} receptor activation can thus be characterised in real time, using fluorometric and fluorescence imaging techniques, including fluorescence microscopy with or without laser confocal methods combined with image analysis algorithms.

Another approach is a high throughput screening assay for compounds active as either agonists or modulators which affect calcium transients. This assay is based around an instrument called a Fluorescence Imaging Plate Reader ((FLIPR®), Molecular Devices Corporation). In its most common configuration, it excites and measures fluorescence emitted by fluorescein-based dyes. It uses an argon-ion laser to produce high power excitation at 488 nm of a fluorophore, a system of optics to rapidly scan the over the bottom of a 96-/384-well plate and a sensitive, cooled CCD camera to capture the emitted fluorescence. It also contains a 96-/384-well pipetting head allowing the instrument to deliver solutions of test agents into the wells of a 96-/384-well plate. The FLIPR assay is designed to measure fluorescence signals from populations of cells before, during and after addition of compounds, in real time, from all 96-/384-wells simultaneously. The FLIPR assay may be used to screen for and characterise compounds functionally active at the hGABA_{B}R1a/GABA_{B}R2 CHO cell line.

A high throughput screening assay, specifically usefull to identify GABA_{B} agonists could consist of an arrangement wherein hGABA_{B}R1a/GABA_{B}R2 CHO cells, are loaded with an appropriate fluorescent dye, incubated with a test compound and after sufficient time to allow interaction (8-24 hours, typically 12-24 hours, in particular 24 hours.) the change in relative fluorescence units measured using an automated fluorescence plate reader such as FLIPR or Ascent Fluoroskan (commercially available from Thermo Labsystems, Brussel, Belgium).

In a further embodiment the functional assay is based on the change in GTPγS binding to the GABA_{B} binding receptor. In particular using a competion bindig assay to determine the displacement of radiolabelled GTPγS. In general, this method to identify GABA_{B}-receptor agonists comprises preparing a membrane fraction from cells expressing the hGABA_{B}R1a/GABA_{B}R2 heterodimer af the present invention, contacting said membrane preparations with the compound to be tested in the presence of radiolabelled GTPγS, under conditions permitting the activation of the GABA_{B} receptor, and detecting GTPγS binding to the membrane fraction. An increase in GTPγS binding in the presence of the compound is an indication that the compound activates the hGABA_{B}R1a/GABA_{B}R2 receptor. A decrease in GTPγS binding in the presence of the compound is an indication that the compound inactivates the hGABA_{B}R1a/GABA_{B}R2 receptor. Preferably this GTPγS binding assay is performed on membrane fractions obtained from the hGABA_{B}R1a/GABA_{B}R2 CHO cell line deposited at the Belgian Coordinated Collections of Microorganisms (3CCM) as CHO-K1 h-GABA-b R1a/R2 clone 20 on Aug. 22, 2003 with the accession number LMBP 6046CB. Further, the conditions permitting the activation of the GABA_{B} receptor comprise the presence of a GABA_{B} receptor agonist, such as for example GABA, baclofen and 3-APPA in the assay system. In particular GABA.

This and other functional screening assays will be provided in the examples hereinafter.

GABA_{B} receptor agonists

In a further aspect the present invention provides GABA_{B} receptor agonists identified using one of the aforementioned screening assays wherein said GABA_{B} receptor agonists are represented by the compounds of formula (I) 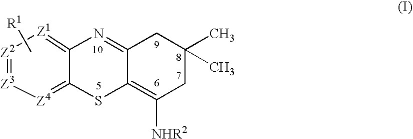
the N-oxide forms, the pharmaceutically acceptable addition salts and the stereochemically isomeric forms thereof, wherein
=Z¹-Z²=Z³-Z⁴=represents a divalent radical selected from the group consisting of
   ═N—CH═CH—N═ (a), ═N—CH═N—CH═ (b), ═CH—N═CH—N═ (c) ═CH—CH═CH—CH═ (d), ═N—CH═CH—CH═ (e), ═CH—N═CH—CH═ (f), ═CH—CH═N—CH═ (g) and ═CH—CH═CH—N═ (h);
R¹ represents hydrogen, halo, hydroxyl, cyano, C₁₋₆alkyl, CF₃, amino or mono- or di(C₁₋₄alkyl)amino;
R² represents hydrogen, C₁₋₆alkyl or hydroxycarbonyl-C₁₋₆alkyl-.

In particular those compounds of formula (I) wherein one or more of the following restrictions apply;
(i) =Z¹-Z²=z³-Z⁴=represents a divalent radical selected from the group consisting of
   ═N—CH═CH—N═ (a), ═N—CH═N-CH═ (b), ═CH—N═CH—N═ (c) and ═CH—CH═CH—CH═ (d);
(ii) R¹ represents halo, amino or mono- or di(C₁₋₄alkyl)amino;
(iii) R² represents butyric acid

Also of interest are those compounds of formula (I) wherein;
(i) R¹ is attached at position Z¹; and/or
(ii) =Z¹-Z²=Z³ -Z⁴=represents (a), (b) or (d), more preferably =Z¹-Z²=Z³-Z⁴=represents (d).

As used in the foregoing definitions and hereinafter, halo is generic to fluoro, chloro, bromo and iodo; C₁₋₄alkyl defines straight and branched chain saturated hydrocarbon radicals having from 1 to 4 carbon atoms such as, for example, methyl, ethyl, propyl, butyl, 1-methylethyl, 2-methylpropyl, 2,2-dimethylethyl and the like; C₁₋₆alkyl defines straight and branched chain saturated hydrocarbon radicals having from 1 to 6 carbon atoms such as, for example, pentyl, hexyl, 3-methylnutyl, 2-methylpentyl and the like.

The pharmaceutically acceptable addition salts as mentioned hereinabove are meant to comprise the therapeutically active non-toxic acid addition salt forms, which the compounds of formula (I), are able to form. The latter can conveniently be obtained by treating the base form with such appropriate acid. Appropriate acids comprise, for example, inorganic acids such as hydrohalic acids, e.g. hydrochloric or hydrobromic acid; sulfuric; nitric; phosphoric and the like acids; or organic acids such as, for example, acetic, propanoic, hydroxyacetic, lactic, pyruvic, oxalic, malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids.

The pharmaceutically acceptable addition salts as mentioned hereinabove are meant to comprise the therapeutically active non-toxic base addition salt forms which the compounds of formula (I), are able to form. Examples of such base addition salt forms are, for example, the sodium, potassium, calcium salts, and also the salts with pharmaceutically acceptable amines such as, for example, ammonia, alkylamines, benzathine, N-methyl-D-glucamine, hydrabamine, amino acids, e.g. arginine, lysine.

Conversely said salt forms can be converted by treatment with an appropriate base or acid into the free acid or base form.

The term addition salt as used hereinabove also comprises the solvates which the compounds of formula (I), as well as the salts thereof, are able to form. Such solvates are for example hydrates, alcoholates and the like.

The term stereochemically isomeric forms as used hereinbefore defines the possible different isomeric as well as conformational forms which the compounds of formula (I), may possess. Unless otherwise mentioned or indicated, the chemical designation of compounds denotes the mixture of all possible stereochemically and conformationally isomeric forms, said mixtures containing all diastereomers, enantiomers and/or conformers of the basic molecular structure. All stereochemically isomeric forms of the compounds of formula (I), both in pure form or in admixture with each other are intended to be embraced within the scope of the present invention.

The N-oxide forms of the compounds of formula (I), are meant to comprise those compounds of formula (I) wherein one or several nitrogen atoms are oxidized to the so-called N-oxide.

The 7,8-dihydro-phenothiazine derivatives of the present invention are generally prepared as described by Nemeryuk M. P. et al., Khimiko-Farmatsevticheskii Zhumal (1985), 19(8), 964-968. In brief, the known ortho-amino substituted (hetero)arene-thiols (II), are condensed with an appropriate 2-bromo-5,5-dimethyl-3-oxo-cyclohex-1-enylamino derivative (III), by heating the two reactants in a suitable solvent, such as ethanol or N-methylpyrrolidone. Standard work-up and purification gives the desired products of formula I (Scheme 1).

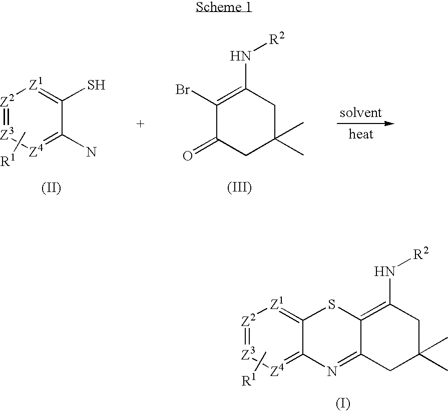

Wherein =Z¹-Z²=Z³-Z⁴=, R¹ and R² are defined as for the compounds of formula (I) hereinbefore.

The appropriate 2-bromo-5,5-dimethyl-3-oxo-cyclohex-1-enylamino derivatives (III) can generally be obtained by amination of 5,5-dimethyl-1,3-cyclohexanedione with the appropriate amine of general formula (IV) under art known amination conditions, followed by bromination with N-bromosuccinimlide (Scheme 2).

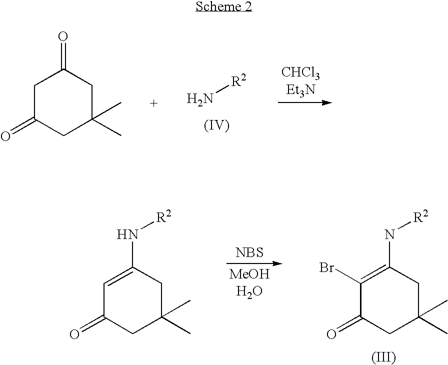

Wherein R² is defined as for the compounds of formula (I) hereinbefore.

For those compounds of formula (I) where R² represents butyric acid, hereinafter referred to as the compounds of formula (I′), the compounds are obtained by condensing the ortho-amino substituted (hetero)arene-thiol (II) with 4-(2-bromo-5,5-dimethyl-3-oxo-cyclohex-1-enylamino)-butyric acid or an ester derivative such as a t-butylester (V) using art known conditions, such as for example by heating the two reactants in a suitable solvent, such as ethanol or N-methylpyrrolidone. Standard work-up and purification gives the desired products, or the ester derivative, which can be hydrolyzed under acidic or basic conditions to give the required butyric acids (I′) (Scheme 3).

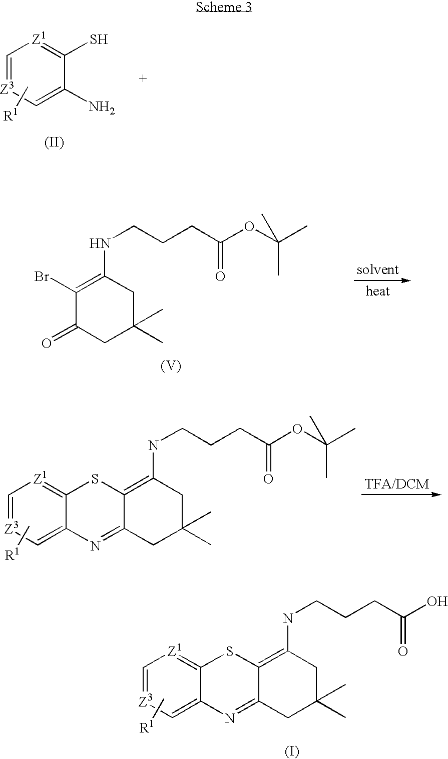

Further examples for the synthesis of compounds of formula (I) using the above mentioned synthesis method is provided in the experimental part hereinafter.

Where necessary or desired, any one or more of the following further steps in any order may be performed:
(i) removing any remaining protecting group(s);
(ii) converting a compound of formula (I) or a protected form thereof into a further compound of formula (I) or a protected form thereof;
(iii) converting a compound of formula (I) or a protected form thereof into a N-oxide, a salt, a quaternary amine or a solvate of a compound of formula (I) or a protected form thereof;
(iv) converting a N-oxide, a salt, a quaternary amine or a solvate of a compound of formula (I) or a protected form thereof into a compound of formula (I) or a protected form thereof;
(v) converting a N-oxide, a salt, a quaternary amine or a solvate of a compound of formula (I) or a protected form thereof into another N-oxide, a pharmaceutically acceptable addition salt a quaternary amine or a solvate of a compound of formula (I) or a protected form thereof.

It will be appreciated by those skilled in the art that in the processes described above the functional groups of intermediate compounds may need to be blocked by protecting groups.

Functional groups which it is desirable to protect include hydroxy, amino and carboxylic acid. Suitable protecting groups for hydroxy include trialkylsilyl groups (e.g. tert-butyldimethylsilyl, tert-butyldiphenylsilyl or trimethylsilyl), benzyl and tetrahydro-pyranyl. Suitable protecting groups for amino include tert-butyloxycarbonyl or benzyloxycarbonyl. Suitable protecting groups for carboxylic acid include C₁₋₆)alkyl or benzyl esters.

The protection and deprotection of functional groups may take place before or after a reaction step.

The use of protecting groups is fully described in ‘Protective Groups in Organic Synthesis’ 3^{rd} edition, T W Greene & P G M Wutz, John Wiley & Sons Inc. (June 1999).

Additionally, the N-atoms in compounds of formula (I) can be methylated by art-known methods using CH₃—I in a suitable solvent such as, for example 2-propanone, tetrahydrofuran or dimethylformamide.

Some of the intermediates and starting materials as used in the reaction procedures mentioned hereinabove are known compounds and may be commercially available or may be prepared according to art-known procedures.

Method of Treatment

The present invention also provides the use of a compound identified as a GABA_{B} receptor activity modulator, using one of the aforementioned assays, in particular the compounds of formula (I) as described hereinbefore, in the manufacture of a medicament for the treatment an indication such as stiff man syndrome, gastroesophogeal reflux, neuropathic pain, incontinence and treatment of cough and cocaine addiction. In particular for use in the manufacture of a medicament to reduce transient lower esophagal sphincter relaxations (TLESR). It is thus an object of the present invention to provide a method for the treatment of a warm-blooded animal, for example, a mammal including humans, suffering from an indication such as stiff man syndrome, gastroesophogeal reflux, neuropathic pain, incontinence and treatment of cough and cocaine addiction, in particular TLESR.

Said method comprising administering to a warm-blooded animal in need thereof an effective amount of a compound identified as a GABA_{B} receptor modulator using a method according to the invention. In particular the systemic or topical administration of an effective amount of a compound according to the invention, to warm-blooded animals, including humans.

Such agents may be formulated into compositions comprising an agent together with a pharmaceutically acceptable carrier or diluent. The agent may in the form of a physiologically functional derivative, such as an ester or a salt, such as an acid addition salt or basic metal salt, or an N or S oxide. Compositions may be formulated for any suitable route and means of administration. Pharmaceutically acceptable carriers or diluents include those used in formulations suitable for oral, rectal, nasal, inhalable, topical (including buccal and sublingual), vaginal or parenteral (including subcutaneous, intramuscular, intravenous, intradermal, intrathecal and epidural) administration. The choice of carrier or diluent will of course depend on the proposed route of administration, which, may depend on the agent and its therapeutic purpose. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. The active compound as defined above may be formulated as suppositories using, for example, polyalkylene glycols, acetylated triglycerides and the like, as the carrier. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolaminc sodium acetate, sorbitan monolaurate, triethanolarnine oleate, etc. Actual methods of preparing such dosage forms are known, or will be apparent, to those skilled in this art; for example, see Gennaro et al., Remington's Pharmaceutical Sciences, Mack Publishing Company, Easton, Pa., 18th Edition, 1990.

The composition or formulation to be administered will, in any event, contain a quantity of the active compound(s) in an amount effective to alleviate the symptoms of the subject being treated.

Dosage forms or compositions containing active ingredient in the range of 0.25 to 95% with the balance made up from non-toxic carrier may be prepared.

For oral administration, a pharmaceutically acceptable non-toxic composition is formed by the incorporation of any of the normally employed excipients, such as, for example, pharmaceutical grades of mannitol, lactose, cellulose, cellulose derivatives, sodium crosscarmellose, starch, magnesium stearate, sodium saccharin, talcum, glucose, sucrose, magnesium, carbonate, and the like. Such compositions take the form of solutions, suspensions, tablets, pills, capsules, powders, sustained release formulations and the like. Such compositions may contain 1%-95% active ingredient, more preferably 2-50%, most preferably 5-8%.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents and the like, such as for example, sodium acetate, sorbitan monolaurate, triethanolamine oleate, triethanolamine sodium acetate, etc.

The percentage of active compound contained in such parental compositions is highly dependent on the specific nature thereof, as well as the activity of the compound and the needs of the subject. However, percentages of active ingredient of 0.1% to 10% in solution are employable, and will be higher if the composition is a solid which will be subsequently diluted to the above percentages. Preferably, the composition will comprise 0.2-2% of the active agent in solution.

Throughout this description the terms “standard methods”, “standard protocols” and “standard procedures”, when used in the context of molecular biology techniques, are to be understood as protocols and procedures found in an ordinary laboratory manual such as: Current Protocols in Molecular Biology, editors F. Ausubel et al., John Wiley and Sons, Inc. 1994, or Sambrook, J., Fritsch, E. F. and Maniatis, T., Molecular Cloning: A laboratory manual, 2nd Ed., Cold Spring Harbor Laboratory Press, Cold Spring Harbor, N.Y. 1989.

This invention will be better understood by reference to the Experimental Details that follow, but those skilled in the art will readily appreciate that these are only illustrative of the invention as described more fully in the claims that follow thereafter. Additionally, throughout this application, various publications are cited. The disclosure of these publications is hereby incorporated by reference into this application to describe more fully the state of the art to which this invention pertains.

### EXPERIMENTAL PART

### I Synthesis of GABA_{B} Agonists

In the procedures described hereinafter the following abbreviations were used: “DIPE” stands for diisopropylether; “EtOAc” stands for ethyl acetate.

For some chemicals the chemical formula was used, e.g. CH₃CN for acetonitrile, NH₃ for ammonia, CH₂Cl₂ for dichloromethane, MgSO₄ for magnesium sulfate, and HCl for hydrochloric acid.

A. Preparation of the Intermediates

### EXAMPLE A.1

Preparation of

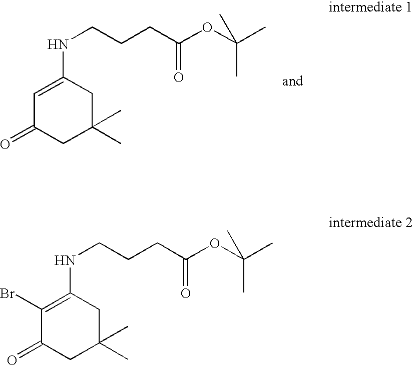

4Aminobutanoic acid 1,1-dimethylethyl ester [50479-22-61 (14 g, 0.087 mol) and 5,5-dimethyl-1,3-cyclohexanedione [126-81-8] (12.26 g, 0.087 mol) were dissolved in trichloromethane (250 ml) and N,N-diethylethanamine (0.5 ml) was added. The reaction mixture was stirred for 3 days and subsequently washed with three portions of 250 ml of water. The organic layer was dried on MgSO₄ and concentrated under reduced pressure. The residue was recrystallised in DIPE/CH₃CN to give 18.6 g (76%) of intermediate 1.

This product was taken up in methanol (250 ml) and water (100 ml). 1-Bromo-2,5-pyrrolidinedione (1 1.8 g, 0.066 mol) was added portionwise over a 30 minutes period. After stirring for an additional hour, 500 ml water was added The mixture was extracted with three portions of dichloromethane. The combined organic layers were dried on MgSO₄ and concenterated under reduced pressure to yield 22 g (92%) of intermediate 2.

In a similar way was also prepared:

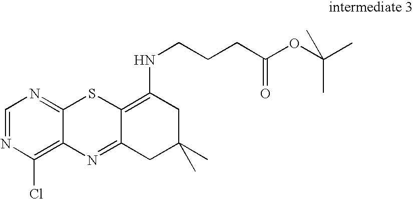

### EXAMPLE A.2

Preparation of

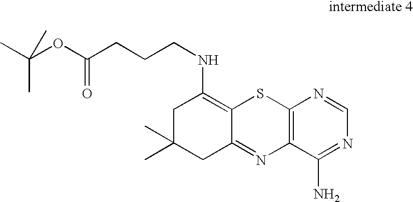

A mixture of 5,6-diamino-4(1H)-pyrimidinethione [2846-89-1](0.0027 mol) and intermediate 2 (0.0027 mol) in ethanol (q.s.) was stirred for 2 hours at 85° C. The reaction mixture was filtered and the solvent was evaporated. The residue was purified by high-performance liquid chromatography. The product fractions were collected and the solvent (CH₃CN) was evaporated. The aqueous layer was extracted with EtOAc. The organic layer was separated, dried (MgSO₄), filtered and the solvent was evaporated, yielding 0.400 g (30%) of intermediate 4.

### EXAMPLE A.3

Preparation of intermediate

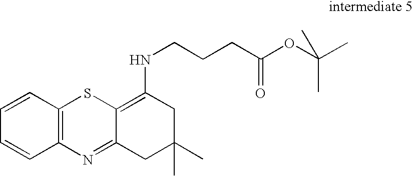

A mixture of 2-arninobenzcncthiol [137-07-5] (0.004 mol) and intermediate 2 (0.004 mol) in 1-methyl-2-pyrrolidinone [872-504] (15 ml) was stirred for 1 hour at 140° C. The reaction mixture was cooled and the layers were separated with EtOAc/H₂O(NH₃). The organic layer was dried (MgSO₄), filtered and the solvent was evaporated. The residue was purified by high-performance liquid chrornatography. The product fractions were collected and the solvent (CH₃CN) was evaporated. The aqueous layer was extracted with EtOAc and then the organic layer was dried (MgSO₄), filtered off and the solvent was evaporated, yielding 0.6 g (40%) of intermediate 5.

B. Prenaration of the Compounds

### EXAMPLE B.1

Preparation of

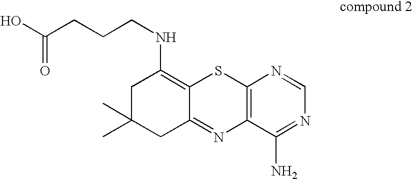

A mixture of intermediate 4 (0.001 mol) in trifluoroacetic acid (5 ml) and dichloromethane (5 ml) was stirred for 1 hour at room temperature. The reaction mixture was dried under a stream of nitrogen. The resulting residue was suspended in diethyl ether. The desired product was filtered off and dried (vacuo) at 30° C., yielding 0.120 g (23%) of trifluoroacetic acid salt of compound 2.

In a similar way were also prepared:

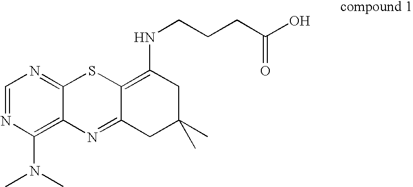

The hydrobromic acid salt of 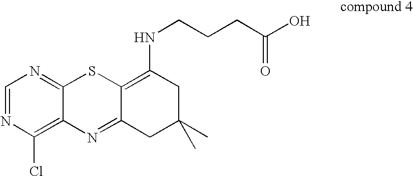
and the trifluoroacetic acid salt of

### EXAMPLE B.2

Preparation of 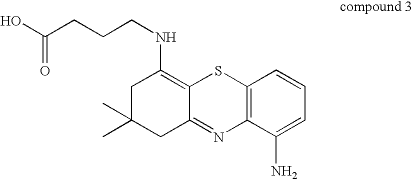
A mixture of intermediate 5 (0.00155 mol) in trifluoroacetic acid (5 ml) and dichloromethane (5 ml) was stirred for 20 hours at room temperature. The reaction mixture was dried under a stream of nitrogen. The resulting residue was solidified in diethyl ether. The desired product was filtered off and dried (vacuo) at 30° C., yielding 0.320 g (67%) of trifluoroacetic acid salt of compound 3.

### II DEVELOPMENT OF GABA_{B}-CHO-K1 CELLS

### Material and Methods

Pennanent transfection of GABA_{B}R1a and GABA_{B}R2 in CHO-K1 cells using Lipofectamine PLUS:

CHO-cells were transfected with hGABABR1a/pcDNA3.1. Monoclonal stable R1a-expressing cells were transfected with hGABABR2/pcDNA3.1 Hygro+. Selection of clones occurred with 800 μg geneticin+800 μg hygromycine/ml.

Menibrane Preparation:

Butyrate-stimulated (5 mM final) cells were scraped, after a short rinse with PBS, in 50 mM TrisHCl pH7.4 and centrifuged at 23500 g for 10 min. at 4° C. The pellet was homogenised in 5 mM TrisHCI pH 7.4 by Ultra-Turrax (24000 rpm) followed by centrifugation at 30000 g for 20 min. at 4° C. The resulting pellet was resuspended in 50 mM TrisHCl pH 7.4 and rehomogenised. Protein concentration was determined using the Bradford method.

GTPγ35S Activation Assay:

10 μg membrane prep was incubated in 250 μl in 20 mM Hepes pH 7.4, 100 mM NaCl, 3 mM MgCl2, 0.25 nM GTPγ35S, 3 μM GDP, 10 μg saponin/ml, with or without 1 mM GABA (basal activity in absence of baclofen) at 37° C. for 20 min. Filtration was carried out onto 96-well GF/B filter plate in Harvester (Packard). Filters were rinsed 6 times with cold 10 mM phosphate buffer pH 7.4, and dried overnight before addition of 30 μl Microscint O, and measurement in Topcount (Packard, 1 min./well).

3H-Agonist Binding:

30-60 μg membrane prep was incubated in 50 mM TrisHCI pH 7.4, 2.5 mM CaCl2, 10 nM 3H-GABA or 20 nM 3H-baclofen in 500 μl at 20° C. Non-specific binding was determined in the presence of 100 μM baclofen. After 90 minutes the mixture was transferred onto 96-well GF/B filterplate by Harvester (Packard). Filters were rinsed 6 times with cold 50 mM TrisHCl pH 7.4, 2.5 mM CaCl2, and dried overnight before addition of 30 μl Microscint O, and measurement in Topcount (Packard, 1 min./well).

### Results

GTPγ³⁵S Activation Assay

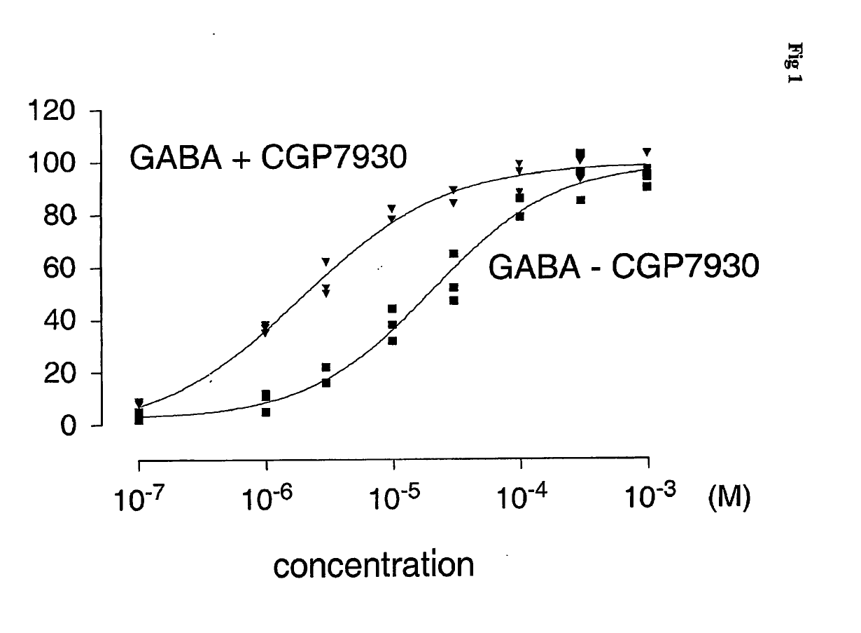

In membranes of stably hGABABR1a-transfected CHO-cells, we measured binding of the antagonist 3H-CGP54626. hGABABR2 was co-transfected in those R1a-clones with the highest antagonist binding. After subcloning stable clones were obtained showing functional activity in GTPγ35S-binding assay upon stimulation of membranes by GABA, wherein said activity was potentiated in the presence of the positive modulator CGP7930 (Urwyler S., et al., 2001, Molecular Pharmacology60:963-971) (FIG. 1).

Agonist Filter Binding Assay

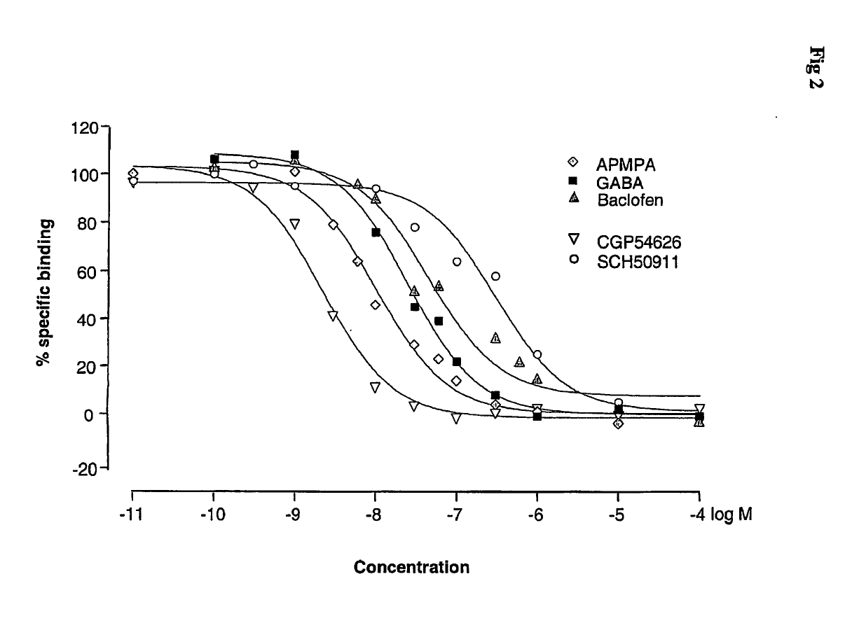

An agonist filter binding assay has been developed in 96-well GF/B filterplate. The IC50 of known agonists and antagonists was determined (FIG. 2). While the stable hGABA_{B}R1a or the transient hGABA_{B}R2 monomeric GABA_{B} receptor expressing cells did not show any binding to the agonists 3H-GABA or 3H-baclofen (data not shown), unexpectedly, in our hGABABR1a/R2 beterodimeric clone agonist binding was detected with both ligands. The Kd for 3H-baclofen, 3H-GABA, and 3H-CGP54626 was determined in saturation experiments and compared well with published results obtained with tissue preparations (table 1).

TABLE 1
³H-baclofen

| | | | |
|---|---|---|---|
| Rat | 132 | nM | (Hill & Bowery, 1981) |
| Dog cortex | 28 | nM | (J&JPRD, 2000) |
| hGABA_{B}R1aR2/CHO | 30 | nM | (our data, n = 2)) |

³H-GABA

| | | | |
|---|---|---|---|
| Rat | 77 | nM | (Hill & Bowery, 1983) |
| Rat | 15-30 | nM | (Cross & Horton, 1988) |
| Pig | 26 | nM | (Facklam & Bowery, 1993) |
| Human | 20-30 | nM | (Cross & Horton, 1988) |
| hGABA_{B}R1aR2/CHO | 10-30 | nM | (our data, n = 6) |

³H-CGP54626

| | | | |
|---|---|---|---|
| Rat | 1.5 | nM | (Bittiger et al., 1993) |
| Pig | 1.35 | nM | (Facklam & Bowery, 1993) |
| hGABA_{B}R1aR2/CHO | 1.5 | nM | (Green et al., 1993) |
| hGABA_{B}R1aR2/CHO | 2.78 | nM | (our data, n = 1) |

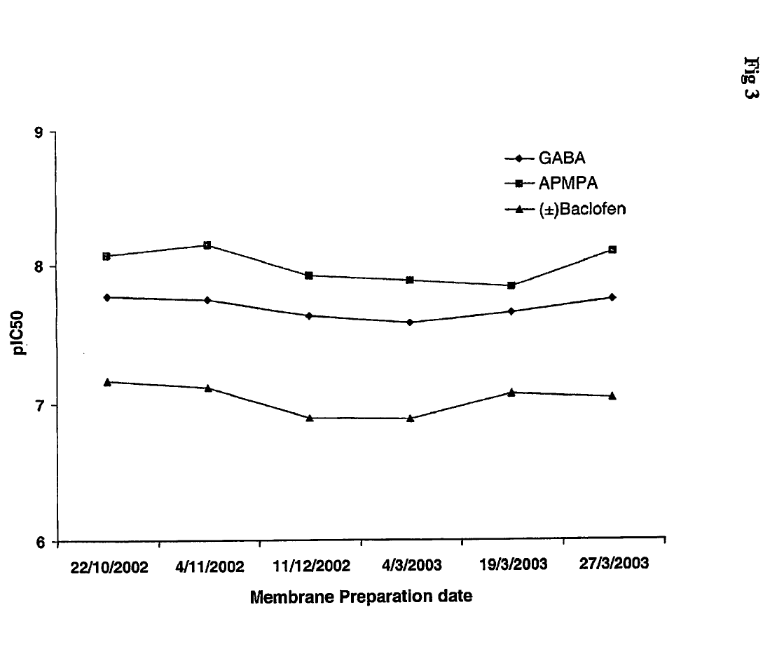

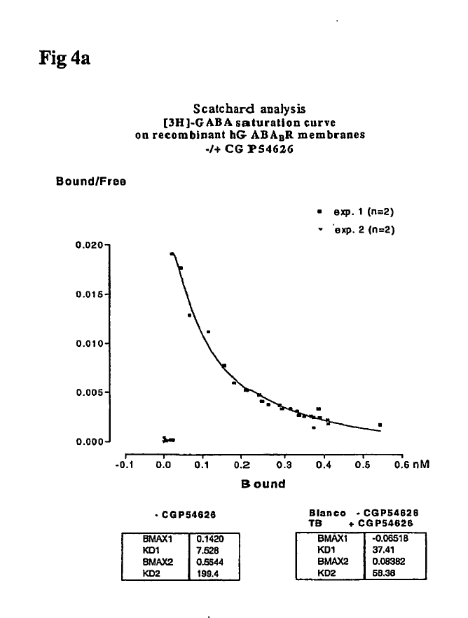

The order of potency for agonists was AMPA>GABA>baclofen, and for antagonists CGP54626>SCH50911 (FIG. 2). The obtained IC50s were reproducible between different membrane preparations (FIG. 3)

Upon full library screening we identified some compounds with binding and signal transduction properties with comparable potencies as the reference compounds GABA and baclofen (table 2).

| | BINDING ASSAY | SIGNAL TRANSDUCTION |
|---|---|---|
| | ³H-GABA binding | GTPγS binding |
| Chemistry | pIC50 | % Effect at 10 μM |
| Reference compounds | | |
| 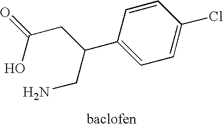 | 6.90775 | 75.7021 |
| 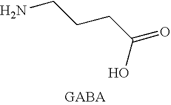 | 8.06026 | 79.2906 |
| HTS hits | | |
| 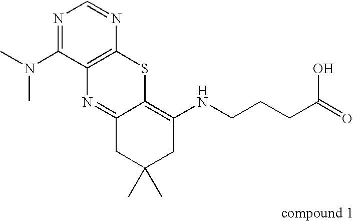 | 7.1875 | 45.7275 |
| 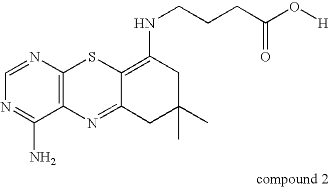 | 6.82 | 40.95 |
| 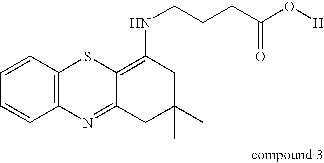 | 6.43 | 24.44 |
| 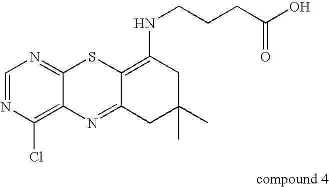 | 6.87 | 61.93 |

Table 2: pIC₅₀ and % effect in the GABA ligand binding, and GTPγS signal transduction assays for reference compounds and HTS hits.

Agonist centrifugation Binding AssayIn an alternative binding assay the non-bound ligand was separated from the membranes by centrifugation instead of filtration. The assay was performed according to the earlier described filter binding assay, with the difference that the non-bound ligand was separated from the membranes by centrifugation in a microcentrifuge at 12500 rpm for 10 minutes. The supernatant was discarded, the pellet was rinsed with washing buffer and dissolved in 200 μl water. Scintillation fluid was added and the bound ³H-GABA measured in Topcount (Packhard, 1 min./well).

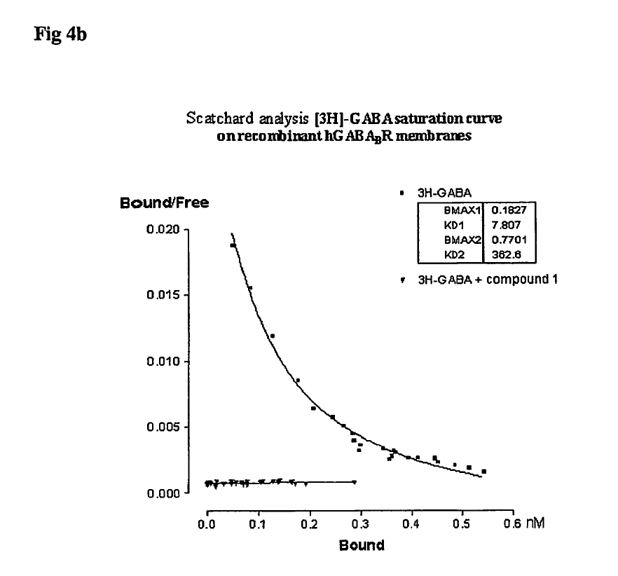

In a saturation assay using increasing concentrations of ³H-GABA (1-400 nM final) I was found that the GABA_{B} receptor expressed by the hGABA_{B}R1a/GABA_{B}R2 CHO cell line, possess a low and a high affinity agonist binding site. Results of the saturation and scatchard analysis are summarized in Table 3. When the saturation assay was preformed in the presence of 10 μM of the GABA_{B} antagonist CGP54626 or one of the GABA_{B} agonist of the present invention (compound 1), the ³H-GABA binding to both the high and the low affinity site was blocked (FIGS. 4a, b).

TABLE 3

| | Mean (n = 5) | SD |
|---|---|---|
| | nM | nM |
| Bmax 1 | 0.19 | 0.05 |
| Kd 1 | 9.4 | 3.1 |
| Bmax 2 | 0.76 | 0.24 |
| Kd 2 | 401 | 224 |

### Discussion

To our knowledge, no earlier reports were made in literature of recombinant hGABA_{B} receptor, showing agonist binding with a high and low affinity binding site in a filter binding assay. An HTS agonist filter binding screen has been developed using 3H-GABA. We found reproducible Ki values for known agonists and antagonists, independent of the membrane preparation.

It has in addition been demonstrated that the recombinant GABA_{B} receptor has two agonist binding sites. One high affinity and one low affinity binding site. It is to be expected that high affinity agonists of the GABA_{B} receptor will ellict a different response compared to the low affinity agonists. Hence, the cell line of the present invention not only allows to identify GABA_{B} receptor agonists, but also provides a useful tool to characterize the nature of the compound receptor interaction.

1. An isolated GABA_{B} receptor protein comprising at least one GABA_{B}R1a subunit and at least one GABA_{B}R2 subunit, characterized in that said GABA_{B} receptor has one high affinity agonist binding site and one low affinity agonist binding site.

2. The GABA_{B} receptor protein according to claim 1 wherein the GABA_{B}R1a subunit is encoded by the oligonucleotide sequence consisting of SEQ ID No.1 and the GABA_{B}R2 subunit is encoded by the oligonucleotide sequence consisting of SEQ ID NO.3.

3. The GABA_{B} receptor protein according to claim 1 wherein said receptor protein is expressed by the hGABA_{B}R1a/GABA_{B}R2 CHO cell line deposited at the Belgian Coordinated Collections of Microorganisms (BCCM) as CHO-K1 h-GABA-b R1 a/R2 clone 20 on Aug. 22, 2003 with the accession number LMBP 6046CB.

4. Use of the GABA_{B} receptor protein according to claim 1 in a method to identify GABA_{B} receptor agonists or antagonists.

5. The hGABA_{B}R1a/GABA_{B}R2 CHO cell line deposited at the Belgian Coordinated Collections of Microorganisms (BCCM) as CHO-K1 h-GABA-b R1a/R2 clone on Aug. 22, 2003 with the accession number LMBP 6046CB.

6. A method to identify whether a test compound binds to a GABA_{B} receptor protein according to claim 1, and is thus a potential agonist or antagonist of the GABA_{B} receptor, said method comprising:
a) contacting cells expressing a functional GABA_{B} receptor, wherein such cells do not normally express the GABA_{B} receptor, with the test compound in the presence and absence of a compound know to bind to the GABA_{B} receptor, and
b) determine the binding of the test compound to the GABA_{B} receptor using the compound known to bind to the GABA_{B} receptor as a reference.

7. A method according to claim 6, wherein the compound known to bind to the GABA_{B} receptor is detectably labeled, and wherein said label is used to determine the binding of the test compound to the GABA_{B} receptor.

8. A method according to claim 7 wherein the compound known to bind to the GABA_{B} receptor is selected from the group consisting of ³H-GABA, ³H-baclofen, ³H-3-APPA, 3H-CGP542626 and ³H-SCH50911.

9. A method to identify GABA_{B} receptor agonists said method comprising,
a) exposing cells expressing a functional GABA_{B} receptor, wherein such cells do not normally express the GABA_{B} receptor, to a labeled agonist of GABA_{B} in the presence and absence of the test compound, and
b) determine the binding of the labeled agonist to said cells,
where if the amount of binding of the labeled agonist is less in the presence of the test compound, then the compound is a potential agonist of the GABA_{B} receptor.

10. A method according to claim 10 wherein the labeled agonist is selected from the group consisting of ³H-GABA, ³H-baclofen and ³H-3-APPA.

11. A method to identify GABA_{B} receptor antagonists said method comprising,
a) exposing cells expressing a functional GABA_{B} receptor, wherein such cells do not normally express the GABA_{B} receptor, to a labeled antagonist of GABA_{B} in the presence and absence of the test compound, and
b) determine the binding of the labeled antagonist to said cells,
where if the amount of binding of the labeled antagonist is less in the presence of the test compound, then the compound is a potential antagonist of the GABA_{B} receptor.

12. A method according to claim 10 wherein the labeled antagonist is selected from the group consisting of ³H-CGP542626 and ³H-SCH50911.

13. A method for identifying a compound as a GABA_{B} receptor agonist, said method comprising;
a) administering the compound to a cellular composition of the cells according to claim 5, in the presence of a detectably labeled GABA_{B} receptor agonist; and
b) determine the binding of the labeled agonist to said cellular composition,
where if the amount of binding of the labeled agonist is less in the presence of the test compound, then the compound is a potential agonist of the GABA_{B} receptor.

14. A method according to claim 13 wherein the cellular composition consists of a membrane fraction of the hGABA_{B}R1a/GABA_{B}R2 CHO cell line deposited at the Belgian Coordinated Collections of Microorganisms (BCCM) as CHO-K1 h-GABA-b R1a/R2 clone on Aug. 22. 2003 with the accession number LMBP 6046CB.

15. A method according to claim 13 wherein the labelled agonist is selected from the group consisting of ³H-GABA, ³H-baclofen and ³H-3-APPA.

16. A method for identifying a compound as a GABA_{B} receptor antagonist, said method comprising;
a) administering the compound to a cellular compositon of the cells according to claim 5, in the presence of a detectably labeled GABA_{B} receptor antagonist; and
b) determine the binding of the labeled antagonist to said cellular composition,
where if the amount of binding of the labeled antagonist is less in the presence of the test compound, then the compound is a potential antagonist of the GABA_{B} receptor.

17. A method according to claim 16 wherein the cellular composition consists of a membrane fraction of the hGABA_{B}R1a/GABA_{B}R2 CHO cell line deposited at the Belgian Coordinated Collections of Microorganisms (BCCM) as CHO-K1 h-GABA-b R1a/R2 clone on Aug. 22, 2003 with the accession number LMBP 6046CB.

18. A method according to claim 16 wherein the labeled antagonist is selected from the group consisting of ³H-CGP542626 and ³H-SCH50911.

19. A method for identifying compounds that have the capability to modulate GABA_{B} receptor activity, said method comprising;
a) contacting cells expressing a functional GABA_{B} receptor, wherein said cells do not normally express a functional GABA_{B} receptor, with at least one reference compound, under conditions permitting the activation of the GABA_{B} receptor;
b) contacting the cells of step a) with a test compound, under conditions permitting the activation of the GABA_{B} receptor, and
c) determine whether said test compound modulates the GABA_{B} receptor activity compared to the reference compound.

20. A method according to claim 19 wherein the capability of the test compound to modulate the GABA_{B} receptor activity is determined using one or more of the functional responses selected form the group consisting of changes in potassium currents, changes in calcium concentration, changes in cAMP and changes in GTPγS binding

21. A method for identifying compounds that have the capability to modulate GABA_{B} receptor activity, said method comprising;
a) contacting a membrane fraction of the cells according to claim 5, with the compound to be tested in the presence of radiolabeld GTPγS, under conditions permitting the activation of the GABA_{B} receptor; and
b) determine GTPγS binding to the membrane fraction,
where an increase in GTPγS binding in the presence of the compound is an indicaton that the compound activates the GABA_{B} receptor activity.

22. A method for identifying compounds that have the capability to modulate GABA_{B} receptor activity, said method comprising;
a) contacting a membrane fraction of the cells according to claim 5, with the compound to be tested in the presence of radiolabeld GTPγS, under conditions permitting the activation of the GABA_{B} receptor; and
b) determine GTPγS binding to the membrane fraction,
where an decrease in GTPγS binding in the presence of the compound is an indicaton that the compound inactivates the GABA_{B} receptor activity.

23. A method according to claim 21 wherein the conditions permitting the activation of the GABA_{B} receptor comprise the presence of a GABA_{B} receptor agonist.

24. A method according to claim 23 wherein the GABA_{B} receptor agonist is selected from the group consisting of GABA, baclofen and 3-APPA.

25. Use of a compounds of formula (I) 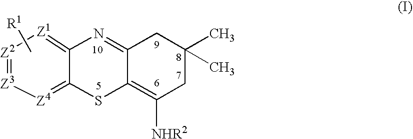
the N-oxide forms, the pharmaceutically acceptable addition salts and the stereochemically isomeric forms thereof, wherein;
=Z¹-Z²=Z³-Z⁴=represents a divalent radical selected from the group consisting of
═N—CH═CH—N═ (a), ═N—CH═N—CH═ (b), ═CH—N═CH—N═ (c) ═CH—CH═CH—CH═ (d), ═N—CH═CH—CH═ (e), ═CH—N═CH—CH═ (f), ═CH—CH═N—CH═ (g) and ═CH—CH═CH—N═ (h);
R¹ represents hydrogen, halo, hydroxyl, cyano, C₁₋₆alkyl, CF₃, amino or mono- or di(C₁₋₄alkyl)amino;
R² represents hydrogen, C₁₋₆alkyl or hydroxycarbonyl-C₁₋₆alkyl-, in the manufacture of a medicament for the treatment of an indication such as stiff man syndrome, gastroesophogeal reflux, neuropathic pain, incontinence and treatment of cough and cocaine addiction.

26. Use of a compound of formula (I) in the manufacture of a medicament to reduce transient lower esophagal sphincter relaxations (TLESR).

27. A compound of formula (I) wherein =Z¹-Z²=Z³-Z⁴=represents (a), (b) or (d), more preferably those compounds of formula (I) wherein =Z¹-Z²=Z³Z4=represents (d).

28. A compound according to claim 27 for use as a medicine.

29. Use of a compound according to claim 27 in the manufacture of a medicament to reduce transient lower esophagal sphincter relaxations (TLESR).
